(19) United States
(12) Patent Application Publication
(10) Pub. No.: US 2015/0366607 A1
(43) Pub. Date: Dec. 24, 2015

(54) SYSTEMS AND METHODS FOR PERFORMING ENDOMETRIAL ABLATION

(71) Applicants: Robin Bek, Campbell, CA (US); Sing Fatt Chin, Pleasanton, CA (US)

(72) Inventors: Robin Bek, Campbell, CA (US); Sing Fatt Chin, Pleasanton, CA (US)

(21) Appl. No.: 14/747,483

(22) Filed: Jun. 23, 2015

Related U.S. Application Data
(60) Provisional application No. 62/016,578, filed on Jun. 24, 2014.

(51) Int. Cl. A61B 18/14

(52) U.S. Cl. CPC A61B 18/1485; A61B 2018/00214

(57) ABSTRACT

An endometrial ablation apparatus for selectively destroying an endometrial lining of an organ in a body including an expandable electrode means for extending the organ and effecting electrical contact with the endometrial lining to be destroyed and an external electrode adapted to contact the outer surface of the body; the expandable electrode containing an electrically non-conductive expansion medium and a variable conductive matrix; and a power source or sources connected to the expandable electrode means and to the external electrode, the power source being adapted to provide radio-frequency electric power to the expandable electrode.

The present invention relates to selective destruction of the endometrium for treating uterine bleeding.

In recent years, a number of therapies have been developed as an alternative to open surgery, often referred to as “least invasive surgery.” Such least invasive surgical procedures are generally characterized by the use of specialized surgical tools in combination with visual or radiographic imaging techniques. The specialized tool is generally inserted through an open body orifice or a small surgical incision, and the tool is then positioned within the body using the imaging technique to allow manipulation of the affected organ or structure.

Least invasive surgery techniques have been applied to the destruction of the inner lining of body organs which provides an alternative to removal of the body organs for treating diseases and abnormal conditions. For example, about 10 million women suffer from heavy menstrual bleeding. Many women begin to experience heavy and/or irregular bleeding in their 30s and 40s, as they begin to get closer to menopause. Heavy periods are more than just a hassle—they take a physical, social, and emotional toll as well.

The destructive treatment of the inner linings has been achieved with chemicals and with various forms of thermal energy such as radiofrequency and microwave heating, cryotherapy, laser and electrosurgery. Radiofrequency and microwave energies have also been applied directly to the linings to generate heat in situ.

### SUMMARY

An endometrial ablation apparatus for selectively destroying an endometrial lining of an organ in a body including an expandable electrode means for extending the organ and effecting electrical contact with the endometrial lining to be destroyed and an external electrode adapted to contact the outer surface of the body; the expandable electrode containing an electrically non-conductive expansion medium that may or maynot be thermally conductive and a variable conductive matrix; and a power source or power sources that are connected to the expandable electrode means and to the external electrode, the power source or power sources being adapted to provide radio-frequency electric power to the expandable electrode.

Advantages of the system may include one or more of the following. The system performs endometrial ablation as a non-invasive alternative to hysterectomy through a unique, radio frequency impedance-based technology, which delivers a uniform RF treatment to lighten or end patients' heavy menstrual bleeding. The system provides fast treatment time, requires no hormonal pretreatment, and can be performed at any time during a woman's cycle—in a hospital or a doctor's office.

### BRIEF DESCRIPTION OF THE DRAWINGS

The instant system for endometrial ablation will be understood by reference to the following detailed description when considered in combination with the accompanying Figures, in which like reference numerals are used to identify like elements throughout this disclosure.

FIG. 1 shows an exemplary radio frequency (RF) endometrial ablation system before inflation of a balloon.

FIG. 2 shows a top view of the RF endometrial ablation system after full expansion using an inflated balloon.

FIG. 3 is a schematic representation of a system using a balloon with a plurality of surface segments as the expandable member.

FIGS. 4A-4C show an exemplary embodiment to the RF ablation system.

FIGS. 5A-5F show various exemplary views of one implementation of FIGS. 4A-4C.

### DESCRIPTION

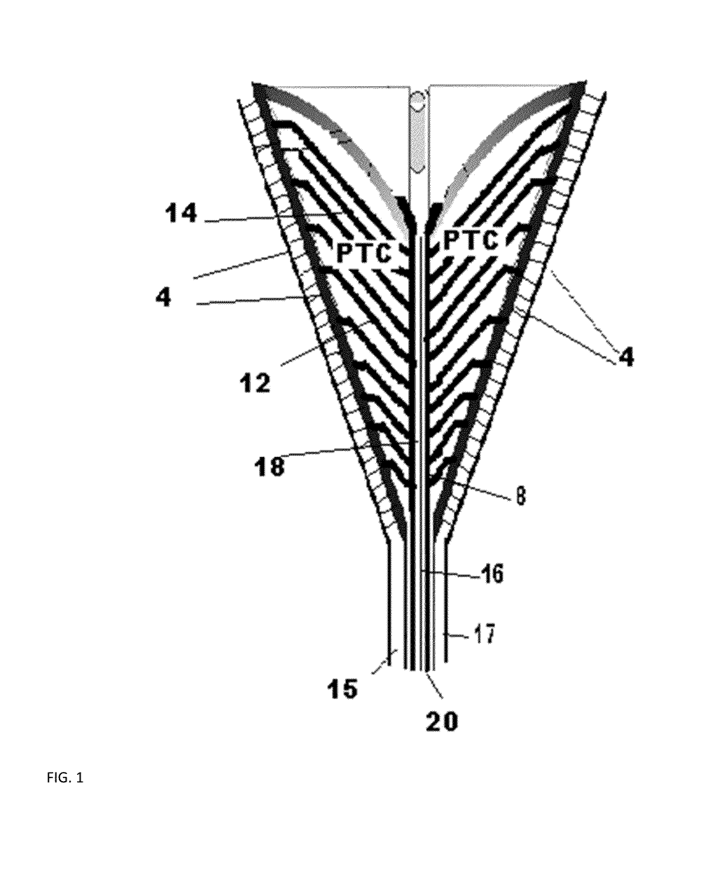

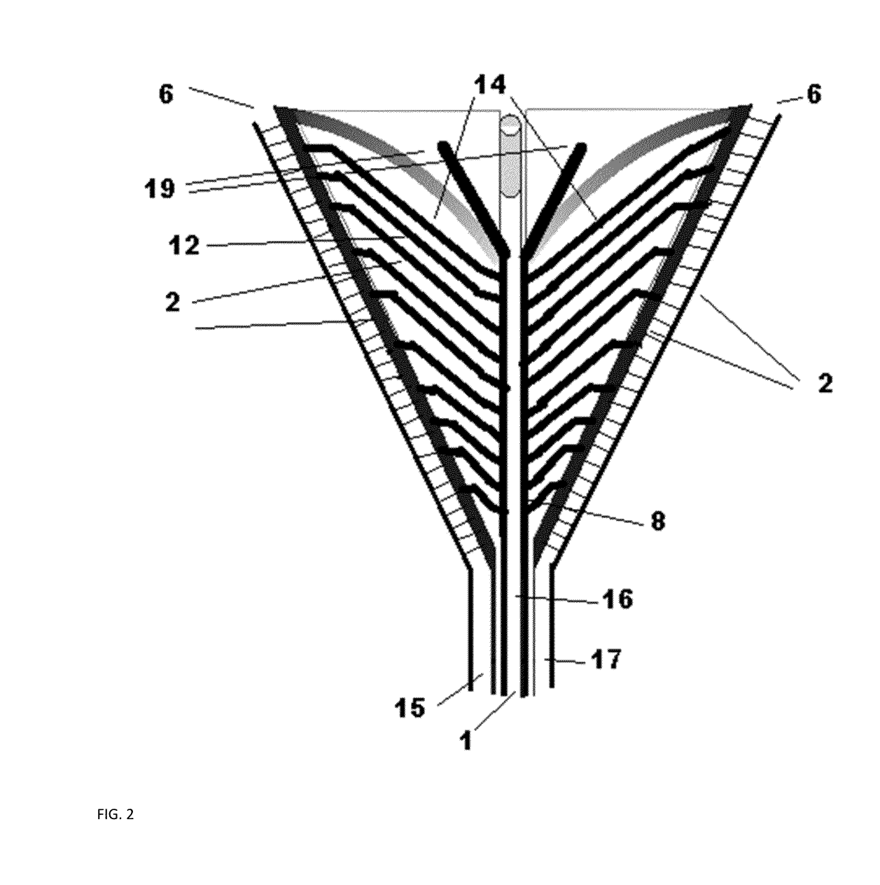

FIG. 1 shows an exemplary radio frequency (RF) endometrial ablation system 1 before inflation of a balloon 14, while FIG. 2 shows a top view of the RF endometrial ablation system 1 after full expansion using an inflated balloon.

Turning now to FIG. 1, the system 1 includes a positive temperature coefficient (PTC) material surrounding an expandable body such as an inflatable balloon 14 or bladder which is inserted by a medical professional into a uterine cavity and subsequently inflating the balloon with fluid (gas or electrically non-conductive liquid) so that it extends the uterine cavity and causes the PTC material to conform to the expanded surface thereof, as shown in FIG. 2. The expandable body 14 can be any material or article which can be compressed or otherwise prepared in a small diameter configuration for insertion through the cervical os 8 and expanded or inflated after the insertion to provide the dilation.

Portions of the balloon 14 extend into the entrance to the fallopian tubes 6 and extend along the entire endometrial surface 12 to the cervical os 8. The balloon is attached to and forms a fluid-tight seal with a main tube 16. Tube 16 encloses a smaller fluid delivery tube 18 and electrical cable 20 containing a single common lead and additional sensor lead(s) such as temperature sensor or fiber optic camera or image sensor. The fluid delivery tube 18 is connected to a source of gas or liquid through a conventional fluid control system (not shown). Manipulable inserter tubes 15 and 17 coexist with tube 18 and can be independently moved in and out to insert and unroll a variably conductive RF treatment surface 2 into a generally triangular treatment surface 2 defined by three points, one at the cervical os 8 and the other two points on each end of the fallopian tubes 6.

Viewing FIGS. 1 and 2 together, the treatment surface 2 provides one or more independent variably conductive RF electrode(s) which expands to conform to the endometrial surface 12 to be treated, dilating and stretching the endometrium to reduce surface folds. Electrical current is passed through or along the treatment surface 2 on the expandable member 14, with the interior of the expandable member 14 being filled with an electrically non-conductive substance such as a fluid. The variably conductive RF electrode(s) must be capable of establishing direct electrical connection or capacitive coupling with the endometrium. The other electrical contact is one or more conventional grounding plates or patches which contact a large area of the patient's skin to complete the circuit.

In one embodiment, the treatment surface is made from a variable conductive matrix. The medial variable conductive matrix can be a polymeric material having a temperature-dependent resistance. Such materials are typically known in the art as polymer-based temperature coefficient materials, and sometimes specifically described as thermally-sensitive resistors or thermistors that exhibit very large changes in resistance with a small change of body temperature. This change of resistance with a change in temperature can result in a positive coefficient of resistance where the resistance increases with an increase in temperature (PTC or positive temperature coefficient material). The scope of the invention also includes a medial variably conductive matrix 140 of a negative temperature coefficient (NTC) material wherein its resistance decreases with an increase in temperature.

Preferably, the variable conductive matrix is a positive temperature coefficient (PTC) material. The PCT material experiences an increase in electrical resistance when its temperature is raised. The higher the coefficient, the greater an increase in electrical resistance for a given temperature increase. One embodiment uses a ceramic material made of barium titanate and other metal oxides excels, which provides a rapid increase in resistance in a narrow temperature range. The PTC ceramic material essentially performs switch delay and stabilization tasks and in addition acts as thermal protection or as self-regulating heating elements.

A suitable variably resistive PTC material can be fabricated from high purity semi-conducting ceramics, for example, based on complex titanate chemical compositions (e.g., BaTiO3, SrTiO3, etc.). The specific resistance-temperature characteristics of the material can be designed by the addition of dopants and/or unique materials processing, such as high pressure forming techniques and precision sintering. Suitable variably resistive or PTC materials are manufactured by a number of sources, and can be obtained, for example from Western Electronic Components Corp., 1250-A Avenida Acaso, Camarillo, Calif. 93012. Another manner of fabricating the medial conductive material is to use a commercially available epoxy that is doped with a type of carbon. In fabricating a substantially thin medial conductive layer in this manner, it is preferable to use a carbon type that has single molecular bonds. It is less preferable to use a carbon type with double bonds which has the potential of breaking down when used in thin layers.

The PCT RF electrode array expands to conform to the contours of each patient's uterine cavity. One embodiment of the system uses a small amount of CO2 to verify cavity integrity prior to performing the procedure. The endometrial ablation system delivers radiofrequency energy until tissue impedance reaches a certain impedance/ohms or when a certain interval of time is reached; on average, the procedure is completed in approximately 60-120 seconds. The electrode array can be retracted for easy removal, leaving the uterine lining desiccated down to the superficial myometrium.

The electrode array passes radiofrequency electric current through the dilated endometrial surface for a time sufficient to destroy the endometrial cells, that is, to elevate the temperature of the endometrium to a temperature of from 45° C. to 90° C., preferably within 10 seconds and maintaining this temperature until the endometrial tissue is destroyed. Optimally, the temperature of the heating is from 55° C. to 65° C.

The expandable balloon or bladder can be an elastomeric polymer such as a natural or synthetic rubber made conductive by mixing the polymer with electroconductive particles such as carbon or conductive metal particles. Alternatively, it can be made conductive by a surface coating of electroconductive material such as an electroconductive gel, or a conductive metal coating on the outer or inner surface of the balloon or bladder wall. Electroconductive coatings can be applied to organic polymer surfaces by conventional vapor deposition, electrical deposition, sputtering and the like.

One exemplary balloon 14 can be a thin, non-extensible polymer film such as a polyester (MYLAR) or other flexible thermoplastic or thermosetting polymer film, for example, having a conductive metal coating on the outer or inner surface thereof. The film forms a non-extensible bladder having a shape and size, in its fully expanded form, which will extend the organ and effecting contact with the endometrial lining to be destroyed. The inner surface of the non-extensible bladder can be coupled to a PCT material. The surface of the expandable member 14 can be open-cell, porous material such as a foam or similar caged network of material which can hold the quantity of the PCT material in solid electrical contact with the opposed endometrial surface. The surface can be coated with or impregnated with the PCT material.

In one embodiment, the PTC matrix is a ceramic layer that can be engineered to exhibit unique resistance vs. temperature characteristics and can maintain a very low base resistance over a wide temperature range, with a dramatically increasing resistance above a specific temperature of the material (sometimes referred to as a Curie point or switching range). The PTC matrix can have a selected switching range between a first temperature (T1) and a second temperature (T2) that approximates the targeted tissue temperature in the contemplated tissue heating objective. The selected switching range, for example, can be any substantially narrow 1°-10° C. range that is determined to be optimal for tissue heating (e.g., any 5° C. range between about 65°-200° C.). A more preferred switching range can fall within the larger range of about 80°-100° C.

In operation, it can be understood that the delivery of RF energy will be conducted through the variably conductive matrix 2 and the endometrial surface 12 to thereby apply RF energy (or active ohmic heating) to tissue contacted by the expandable body or balloon 14. After the engaged tissue is elevated in temperature by such active RF heating, the mass of the endometrial surface 12 will be modulated in temperature at or about the desired temperature range and thereafter, the variable conductive matrix will conduct or radiate thermal effects to the engaged tissue. In this manner, the increase in temperature of the variably resistive matrix is typically caused by the transient high temperature of tissue that is caused by active RF heating of the tissue.

The expandable body 14 engages and compresses the variable conductive matrix 2 tightly with the endometrial tissue and thereafter applying active RF energy to the tissue to maintain a selected temperature for a selected time interval. For example, the instrument is provided with a working end that carries a medial variably conductor matrix 2 that has a switching range at or about 90° C. at which its resistance increases greater than about 5% (and can be as much as 1,000,000% or more) above its low base resistively with a change in temperature of about 5° C. or less.

With the balloon 14 engaging tissue 12, the operator actuates a switch that delivers RF energy from the voltage (RF) source to the interior conductor. At ambient tissue temperature, the low base resistance of the medial conductive matrix 2 allows unimpeded RF current flow from the voltage source 150 through the engagement surface 12 and tissue to return electrical lead that is coupled to the ground pad or the bipolar return. It can be understood that the engaged tissue initially will have a substantially uniform impedance to electrical current flow, which will increase substantially in proximity to engagement surface 12 as the engaged tissue loses moisture due to the active RF delivery. Following an arbitrary time interval, the impedance of tissue proximate to engagement surface 12 typically will be elevated, and the higher tissue temperature will instantly conduct heat to the medial PTC matrix 2. In turn, the medial PTC layer 2 will reach its switching range and terminate RF current flow from its conductor to the engagement surface 12. Such automatic reduction of active RF energy application will prevent any substantial dehydration of tissue. By thus maintaining the desired level of moisture in tissue proximate to the surface 2, the working end can more effectively apply energy to the tissue. Such energy application can extend through thick engaged tissue volumes while causing very limited collateral thermal effects. Thereafter, as the temperature of tissue proximate to engagement surface 12 falls by thermal relaxation and the lack of an RF energy density, the temperature of the medial conductive matrix 2 will thus fall below the threshold of the selected switching range. This effect, in turn, will cause RF current to again flow to the engaged tissue to again increase the tissue temperature by active RF heating. By the above-described mechanisms of causing the medial variably resistive matrix 2 to hover about its selected switching range, the actual RF energy applied to the engaged tissue can be precisely modulated to maintain the desired temperature in the tissue.

Of particular interest, in one embodiment, the polymer matrix that comprises the medial conductor portion 2 is doped with materials to resistively heat the matrix as RF energy flow there through is reduced. Thus, the thermal mass of the PCT matrix which are elevated in temperature can deliver energy to the engaged tissue by means of greater passive conductive heating—at the same time RF energy delivery causes lesser active tissue heating. This balance of active RF heating and passive conductive (or radiative) heating can maintain the targeted temperature for any selected time interval.

In summary, one method provides for the delivery of RF energy from a voltage source 150 to a tissue facing surface 2 through a thermally-sensitive resistor material wherein the resistor material has a selected switching range that approximates a targeted temperature for tissue heating. In operation, the working end automatically modulates active RF energy density in the tissue as the temperature of the engaged tissue conducts heat back to the thermally-sensitive resistor material 2 to cause its temperature to reach the selected switching range. In this range, the RF current flow will be reduced, with the result being that the tissue temperature can be maintained in the selected range without the need for thermocouples or any other form of feedback circuitry mechanisms to modulate RF power from the source. The system allows for immediate modulation of actual RF energy application along the entire length of the endometrium, which is to be contrasted with prior art instruments that utilize a temperature sensor and feedback circuitry. Such sensors or thermocouples measure temperature only at a single location in the endometrium, which typically will not be optimal for energy delivery over the length of the endometrium. Such temperature sensors also suffer from a time lag. Further, such temperature sensors provide only an indirect reading of actual tissue temperature—since a typical sensor can only measure the temperature of the electrode.

In one embodiment, at least one of the inserter tubes 15 and 17 carries a fiberoptic camera 19 on top to allow an operator the medical professional to view the placement of the ablation device 1. A fiberoptic microendoscope is arranged through at least one of the tubular inserter 15 or 17. The endoscope has a distal end arranged near the distal end of the inserter 15/17, and a proximal end, which may be fitted with a viewing eyepiece or camera or the like to ensure that the tubes 15, 16 and 17 do not accidentally pierce the endometrium.

In one embodiment without visual feedback of the placement of the device 1, CO2 pressure containment in the balloon 14 can be used to confirm that the device 1 has not accidentally pierced the endometrium. In another embodiment, flow of gas into the balloon 14 can provide placement feedback to the operator. In yet another embodiment, visual, pressure, or flow of gas can be used in combination to indicate proper placement of the device 1.

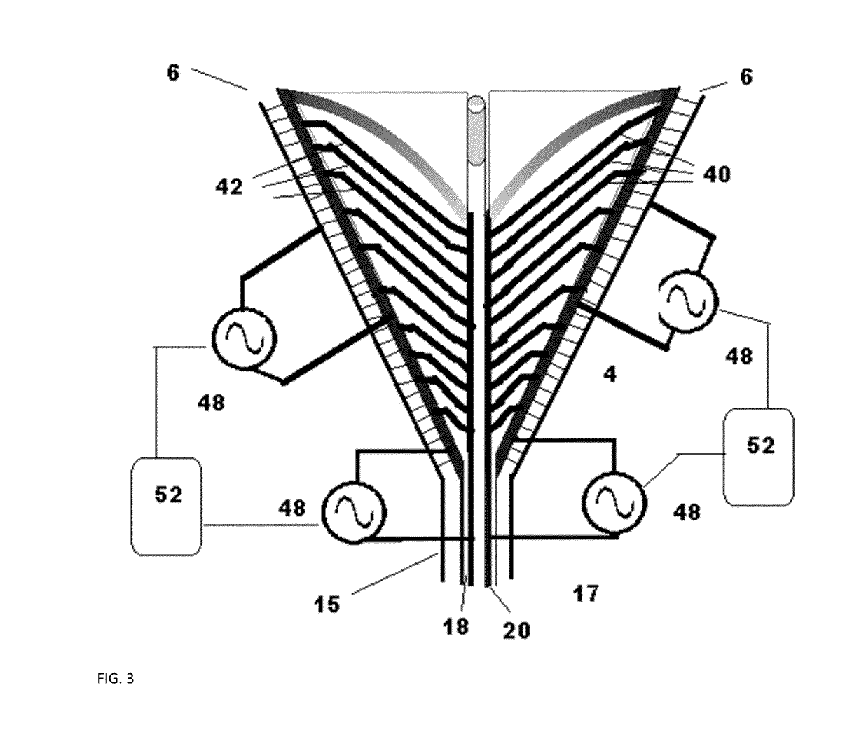

FIG. 3 is a schematic representation of a system using a balloon with a plurality of surface segments as the expandable member, each with a variably resistive surface. In this embodiment the balloon or inextensible bladder has a segmented variably resistive surface on either the inner or the outer surface thereof to permit controlled delivery of energy to each segment 40. Each segment 40 is electrically connected through conventional leads (not shown) to the power source. Each conductive segment 40 also has a separate RF power supply which is connected through conventional leads (not shown) to a power switch matrix. The segmented embodiment of FIG. 3 can be used in a monopolar or bipolar mode. The surface of the bladder 44 has attached thereto the conductive electrode segments 40. Electrical leads separately connect each electrode segment to a separate power supply 48. Electrical leads separately connect each segment 40 with its own impedance sensor 48 (ohm meter) to the controller 52 where each temperature sensor is sampled by sensing the impedance from each power supply. In response, the controller 52 applies power to each conductive segment as required to maintain the temperature of the corresponding endometrium segment within the desired range during the treatment. It will be readily apparent to a person skilled in the art that the power can be applied simultaneously, sequentially or any other desired pattern to the electrode segments.

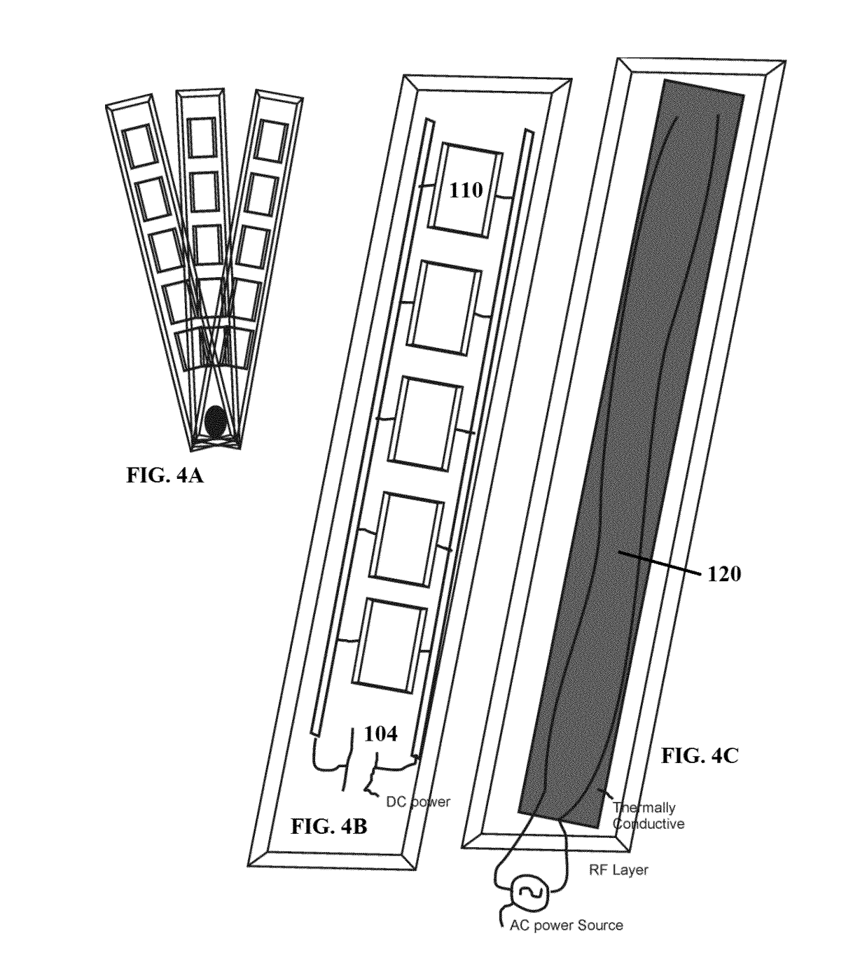

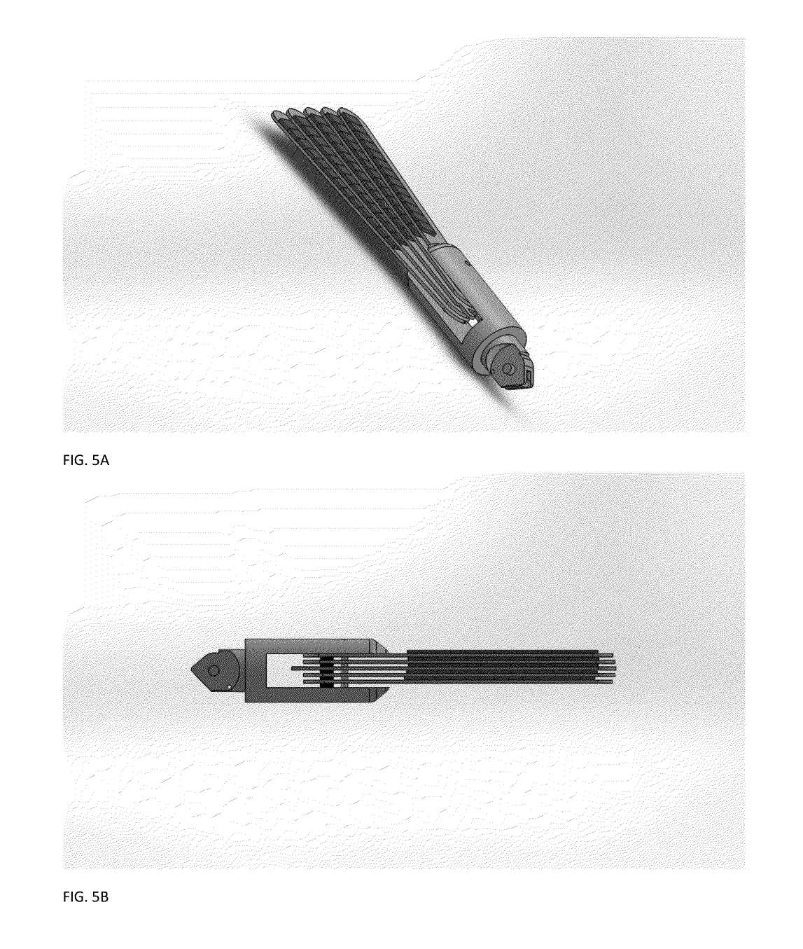

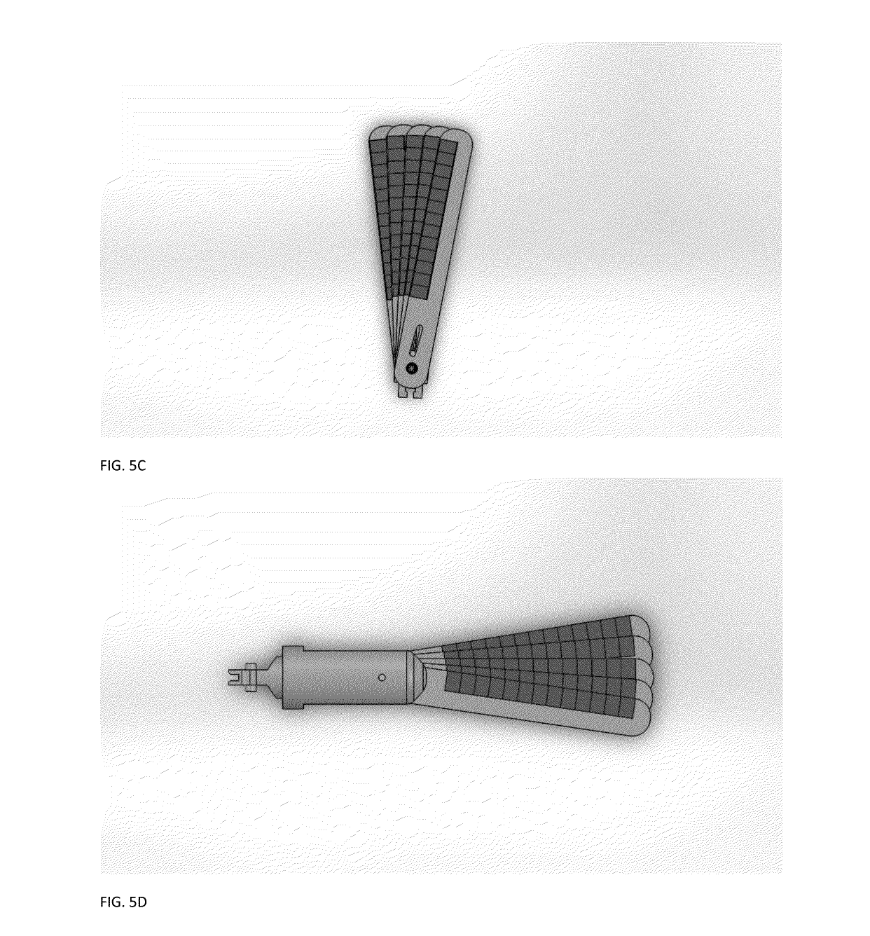

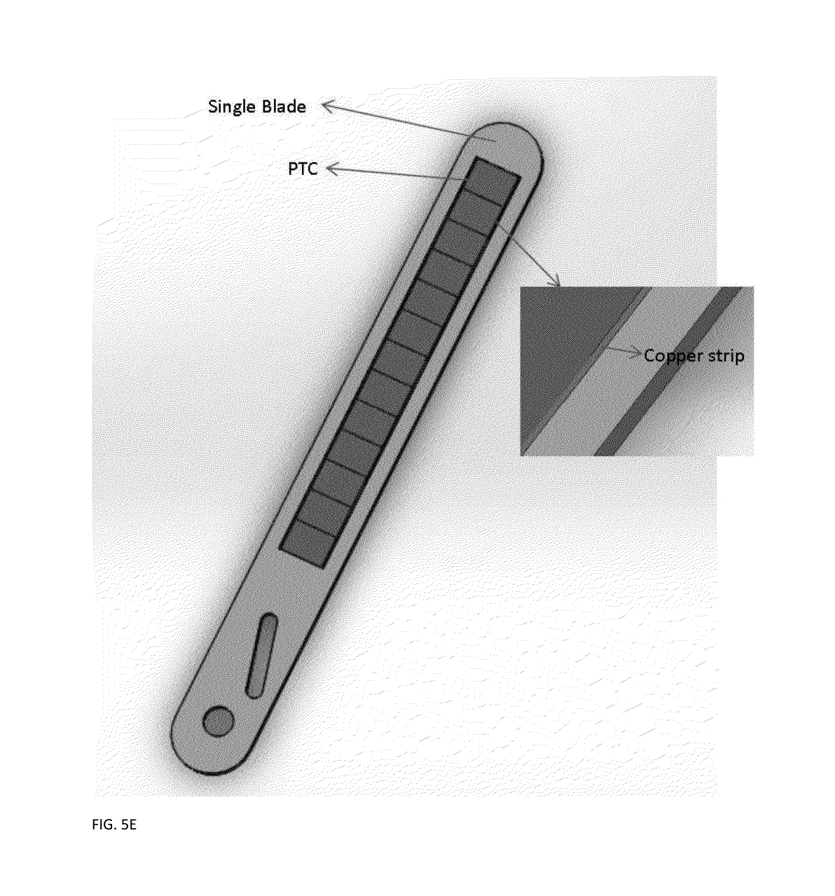

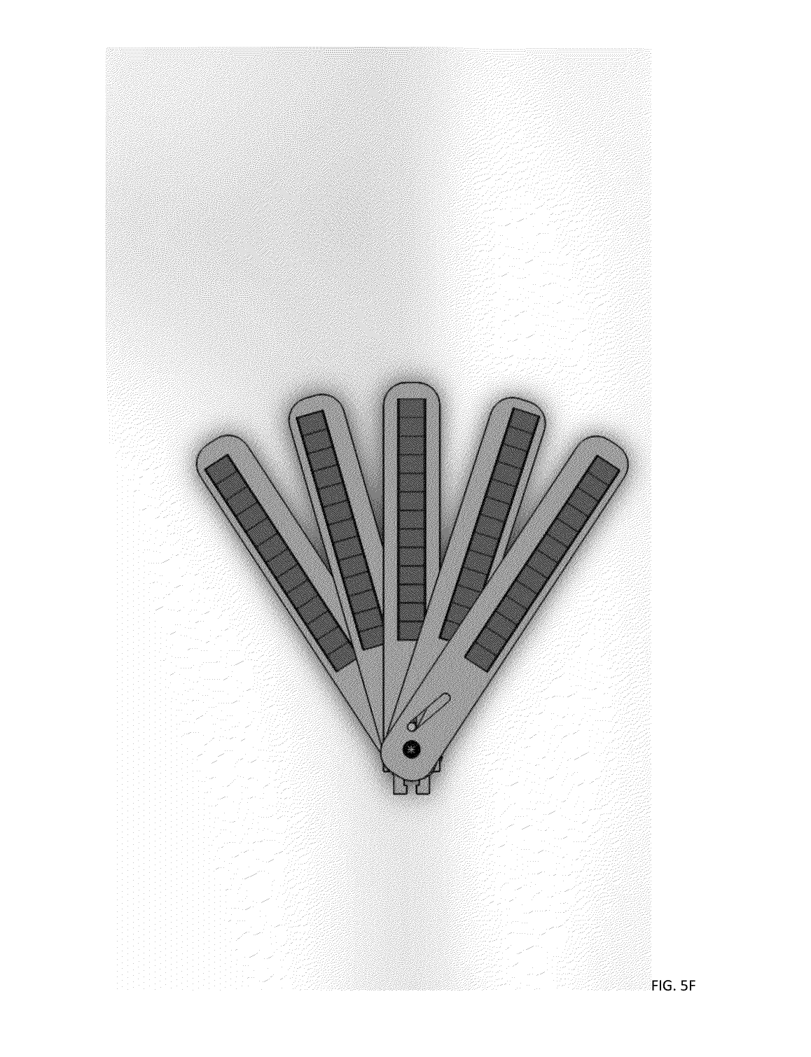

FIGS. 4A-4C shows a new electrode configuration with a more consistent ablation while FIGS. 5A-5F show various exemplary views of FIGS. 4A-4C. FIG. 4A shows a plurality of panels, each of which is shown in FIGS. 4B (front view) and 4C (rear view). The electrode is composed of multiple PTC 110 devices that are connected to an AC or DC source of ENERGY 104. The rear includes a non-conductive electrical material 120 that is thermally conductive. These PTC electrodes heat up and respond to changes in loading when brought into the proximity of target tissue. There are also electrodes that provide RF energy that are also used to ablate tissue. These can be configured as bipolar or monopolar electrodes. The RF electrode and the PTC are on opposite sides of the same electrode that is separated by a membrane that is thermally conductive and electrically non-conductive. One embodiment allows the heat that the PTCs generates to move across the membrane. This will allow the electrode to automatically adapt to the thermal environment and maintain a consistent ablation.

While all the strategies may be used, in a temperature-controlled ablation, the system may use the average temperature of the impedance sensors to control the power output, voltage output or current output. Alternatively, high and/or low readings may be removed from the calculation. Optionally, one may employ a power voltage control algorithm which operates differently while it is ramping up to a target temperature, as compared to its operation when it is at or near the target temperature. In accordance with another embodiment of the invention, in ramping mode, a ramping mode power control algorithm applies the full power of the system, reduced by an amount, if any, which causes the system to implement a maximum temperature increase rate of 2° C./second. In this embodiment, this ramp rate may be reduced as average temperature measured by the temperature transducers in the probe's ablation surfaces approach the target temperature. When the probe average temperature is below, and within, for example, 0.5° C. of the target temperature, the power control algorithm switches to target power control mode where power is moderated and adjusted to maintain the desired temperature. Target power control delivers power/voltage in proportion to the small differences between the thermocouple average and the target temperature. After the algorithm has switched from ramping to target power control mode, the system may be set to never switch back to ramping mode until the RF power is turned off. That means once target temperature is achieved the amount of RF Power delivered is only to maintain target temperature.

As alluded to above, manual (or power) control mode simply delivers the amount of power to the electrode array that has been set as the target power.

In both temperature and manual modes, the amount of ablation time may be controlled by a foot pedal that is used to start and stop the RF delivery.

While illustrative embodiments of the invention have been described, it is noted that various modifications will be apparent to those of ordinary skill in the art in view of the above description and drawings. Such modifications are within the scope of the invention which is limited and defined only by the following claims.

What is claimed is:

1. An endometrial ablation apparatus for selectively destroying an endometrial lining of an organ in a body, comprising:
an expandable electrode means for extending the organ and effecting electrical contact with the endometrial lining to be destroyed and an external electrode adapted to contact the outer surface of the body; the expandable electrode containing an electrically non-conductive expansion medium and a variable conductive matrix; and
a power source connected to the expandable electrode means and to the external electrode, the power source being adapted to provide radio-frequency electric power to the expandable electrode.

2. An endometrial ablation apparatus of claim 1, wherein the power source passes current from the expandable electrode through the endometrial lining, selectively heats the endometrial lining to a temperature within the range of from about 45° C. to 90° C.

3. An endometrial ablation apparatus of claim 1, wherein the frequency is within the range of from about 100 kHz to about 100 MHz. The PTC elements could also be driven by a DC signal.

4. An endometrial ablation apparatus of claim 1, wherein the expandable electrode is a balloon adapted to be connected to an expansion fluid inlet means, the balloon being filled with a gas or an electrically non-conductive liquid.

5. An endometrial ablation apparatus of claim 1, wherein the variable conductive matrix comprises a positive temperature coefficient (PTC) material.

6. An endometrial ablation apparatus of claim 1, wherein the variable conductive matrix comprises a complex titanate chemical composition.

7. An endometrial ablation apparatus of claim 6, wherein specific resistance-temperature characteristics of the material is varied by adding dopants and/or materials processing including high pressure forming or sintering.

8. An endometrial ablation apparatus of claim 1, wherein the variable conductive matrix comprises a negative or positive temperature coefficient (NTC) material.

9. An endometrial ablation apparatus of claim 1, comprising a back portion with a non-conductive electrical material that is thermally conductive.
